(19) United States
(12) Patent Application Publication
(10) Pub. No.: US 2019/0269102 A1
(43) Pub. Date: Sep. 5, 2019

(54) HABITAT DETERMINATION METHOD, INFORMATION PROCESSING APPARATUS, AND COMPUTER-READABLE RECORDING MEDIUM RECORDING HABITAT DETERMINATION PROGRAM

(71) Applicant: FUJITSU LIMITED, Kawasaki-shi (JP)

(72) Inventors: Hitoshi Komoriya, Machida (JP); Tomoko Nagano, Isehara (JP); Masatoshi Takenouchi, Hadano (JP)

(73) Assignee: FUJITSU LIMITED, Kawasaki-shi (JP)

(21) Appl. No.: 16/417,052

(22) Filed: May 20, 2019

Related U.S. Application Data
(63) Continuation of application No. PCT/JP2016/085620, filed on Nov. 30, 2016.

(51) Int. Cl. A01K 29/00; A01K 67/00; G06Q 50/02

(52) U.S. Cl. CPC A01K 29/00; G06Q 50/02; A01K 67/00

(57) ABSTRACT

A habitat determination method includes: calculating, by a computer, a first indicator, which is a number of trees per predetermined area with respect to a tree which allows tree mammals to inhabit; and determining, by the computer, according to the calculated first indicator and a characteristic of each tree mammal, whether or not each tree mammal is inhabitable.

### CROSS-REFERENCE TO RELATED APPLICATION

This application is a continuation application of International Application PCT/JP2016/085620 filed on Nov. 30, 2016 and designated the U.S., the entire contents of which are incorporated herein by reference.

### FIELD

The embodiments discussed herein are related to a habitat determination method, an information processing apparatus, and a habitat determination program.

### BACKGROUND

Readily available statistical data or the like is used when determining whether or not an animal inhabits a target area.

Japanese Laid-open Patent Publication No. 2015-139439, Japanese Laid-open Patent Publication No. 2015-008656, and Japanese Laid-open Patent Publication No. 2011-165112 are disclosed as related art.

### SUMMARY

According to an aspect of the embodiments, a habitat determination method includes: calculating, by a computer, a first indicator, which is a number of trees per predetermined area with respect to a tree which allows tree mammals to inhabit; and determining, by the computer, according to the calculated first indicator and a characteristic of each tree mammal, whether or not each tree mammal is inhabitable.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of functional blocks of a habitat determination apparatus according to a first embodiment;

FIG. 2 is a diagram illustrating an example of a vegetation database (DB) according to the first embodiment;

FIG. 3 is a diagram illustrating an example of a recording DB according to the first embodiment;

FIG. 4 is a diagram illustrating an example of an animal feature DB;

FIG. 5 is a diagram illustrating an example of a tree age DB according to the first embodiment;

FIG. 6 is a diagram illustrating an example of a determination result DB according to the first embodiment;

FIG. 7 is a diagram illustrating an example of a determination technique according to related art;

FIG. 8 is a flowchart illustrating an example of a pre-determination process;

FIG. 9 is a flowchart illustrating an example of a habitat determination process according to the first embodiment;

FIG. 10 is a diagram illustrating an example of a recording DB according to a second embodiment;

FIG. 11 is a diagram illustrating an example of a tree age DB according to the second embodiment;

FIG. 12 is a diagram illustrating an example of a determination result DB according to the second embodiment;

FIG. 13 is a flowchart illustrating an example of a habitat determination process according to the second embodiment;

FIG. 14 is a diagram illustrating an example of a vegetation DB according to a third embodiment;

FIG. 15 is a diagram illustrating an example of a determination result DB according to the third embodiment;

FIG. 16 is a flowchart illustrating an example of a habitat determination process according to the third embodiment; and

FIG. 17 is a diagram illustrating an example of a computer that executes a habitat determination program.

### DESCRIPTION OF EMBODIMENTS

For example, distribution data of the population density of a selected biological species in an evaluation target region is created, using the traffic volume of a road calculated from map data including roads and buildings in the evaluation target region, and the suitability of the evaluation target region as a habitat is evaluated. For example, a technology is provided, in which, on the basis of green space data according to the categorization and the distribution situation of green spaces on the ground surface, indicators such as a green space continuity indicator and a green space grouping indicator are calculated and, on the basis of each indicator and the green space data, the habitat suitability for a biological species is estimated. Furthermore, for example, a technology for extracting a forest patch, which is a land section covered by trees, from remote sensing data of a target area, and calculating a habitat suitability index for a forest-living organism corresponding to the forest patch is also provided.

Incidentally, tree mammals such as squirrels, possums, flying squirrels, and gliders themselves rarely make a nest to protect themselves from natural enemies such as owls and, accordingly these tree mammals use existing holes on old trees as nest holes. However, since the above-mentioned technologies using statistical data and the like do not take into consideration the presence or absence of an environment that can be used as a nest hole by the tree mammal, the accuracy of determining whether or not the tree mammal inhabits is low in some cases. Meanwhile, according to the method of statistically processing data of actually measured witness counts, masses of feces, bite marks on plants, and number of removed individuals for prediction, the accuracy of determination can be improved, but it takes a lot of work and time to actually measure animals.

A habitat determination method, a habitat determination apparatus, and a habitat determination program capable of improving the accuracy of determining whether or not a targeted animal inhabits may be provided.

Hereinafter, embodiments of a habitat determination apparatus disclosed in the present application will be described in detail with reference to the drawings. Note that the present invention is not limited by these embodiments. Furthermore, the respective embodiments indicated below may be appropriately combined within a range not causing conflict.

### First Embodiment

In the present embodiment, a tree mammal to be subjected to a habitat determination process inhabits, for example, forests of eucalyptus. Eucalyptus, which grows to a tall tree, has a deft in the trunk as the tree trunk grows thicker from tree age of about 30 years and produces a tree hole utilized by a tree mammal as a hiding place. For example, the size of the hole on the eucalyptus tree is enlarged with tree age.

Incidentally, also tree mammals sometimes have different body sizes depending on the type and some tree mammals are not allowed to use the holes on trees as nest holes depending on the size of the holes. For example, when the tree age is low and the entrance to the tree hole is small, a smaller type of tree mammal such as a yellow-bellied glider (sometimes abbreviated as “YG” below) uses such a hole as a nest hole. On the other hand, a larger type of tree mammal such as a greater glider (sometimes abbreviated as “GG” below) is not allowed to use a hole on a tree with low tree age as a nest hole. Conversely, a smaller type of tree mammal such as the YG will not use the hole of a tree having the larger entrance due to the higher tree age.

[Functional Blocks]

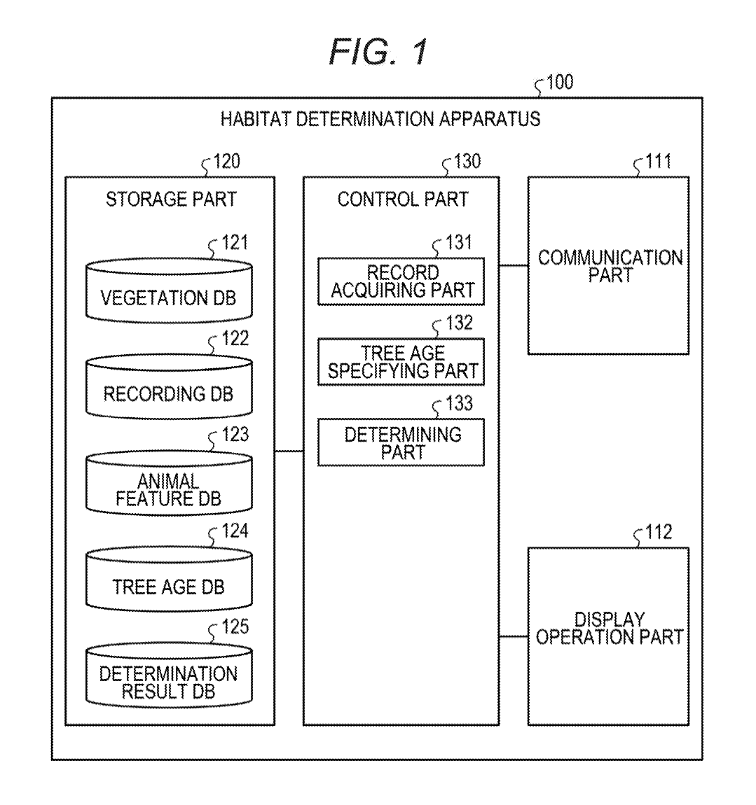

The present embodiment thus will describe a configuration for performing a habitat determination process by reflecting a tree size according to tree age in addition to a first indicator, which is the number of trees per predetermined area. First, the functional configuration of the present embodiment will be described. FIG. 1 is a diagram illustrating an example of functional blocks of a habitat determination apparatus according to the first embodiment. As illustrated in FIG. 1, the habitat determination apparatus 100 has a communication part 111, a display operation part 112, a storage part 120, and a control part 130. The habitat determination apparatus 100 is implemented by an instrument such as a computer and may have various sorts of functional units included in a known computer, for example, various sorts of functional units such as an input device and an audio output device, in addition to the functional units illustrated in FIG. 1.

The communication part 111 controls communication with an external database server (not illustrated), a terminal of a user (not illustrated), and the like by way of a network N (not illustrated) regardless of whether the network N is wired or wireless. The display operation part 112 displays information output from the control part 130. The display operation part 112 accepts an operation by a user (not illustrated) and outputs the accepted operation to the control part 130.

The storage part 120 stores, for example, a program executed by the control part 130, various sorts of data and the like. The storage part 120 also has a vegetation DB 121, a recording DB 122, an animal feature DB 123, a tree age DB 124, and a determination result DB 125. The storage part 120 corresponds to a semiconductor memory element such as a random access memory (RAM), a read only memory (ROM), and a flash memory, or a storage apparatus such as a hard disk drive (HDD).

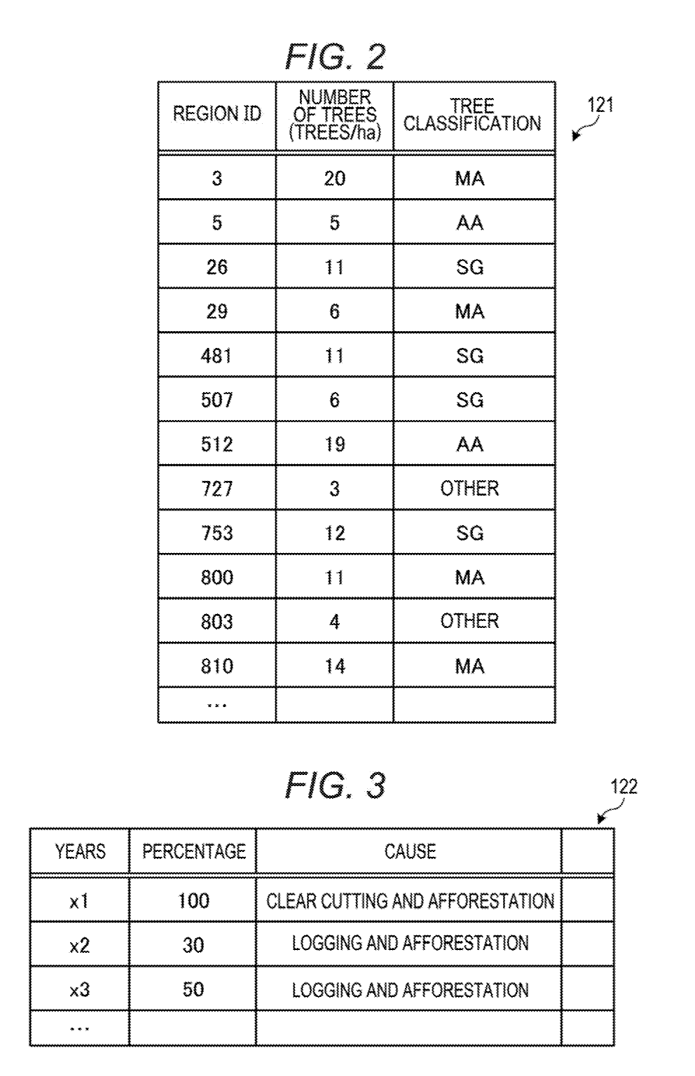

The vegetation DB 121 stores the situation of trees at each time point in each region. FIG. 2 is a diagram illustrating an example of a vegetation DB according to the first embodiment. As illustrated in FIG. 2, the vegetation DB 121 stores “number of trees” and “tree classification” in association with “region ID”. The information stored in the vegetation DB 121 is, for example, information acquired by a record acquiring part 131 to be described later, which is disclosed in a vegetation map or the like released by the environmental authorities.

In FIG. 2, “tree classification” stores the classification of a tree planted in a target region. “MA”, “AA” and “SG” in FIG. 2 are abbreviations for “mountain ash”, “alpine ash”, and “shining gum”, which are classifications of trees, respectively. In the present embodiment, it is assumed that one type of trees is planted in one target region.

Returning to FIG. 1, the recording DB 122 stores records on logging and the like in each region. FIG. 3 is a diagram illustrating an example of the recording DB according to the first embodiment. For example, the recording DB 122 has one table for each targeted region ID. The information stored in the recording DB 122 is, for example, information acquired by the record acquiring part 131 to be described later, which is disclosed in logging records or the like released by the environmental authorities.

As illustrated in FIG. 3, the recording DB 122 stores “years”, “percentage”, and “cause” in association with each other. “Years” stores how many years ago from the current time point the cause such as logging occurred. In the present embodiment, it is assumed that the years are sequentially stored from the oldest one. “Percentage” indicates the percentage of trees lost in the target region due to the cause. “Cause” indicates the contents of logging or the like that has occurred in the target region.

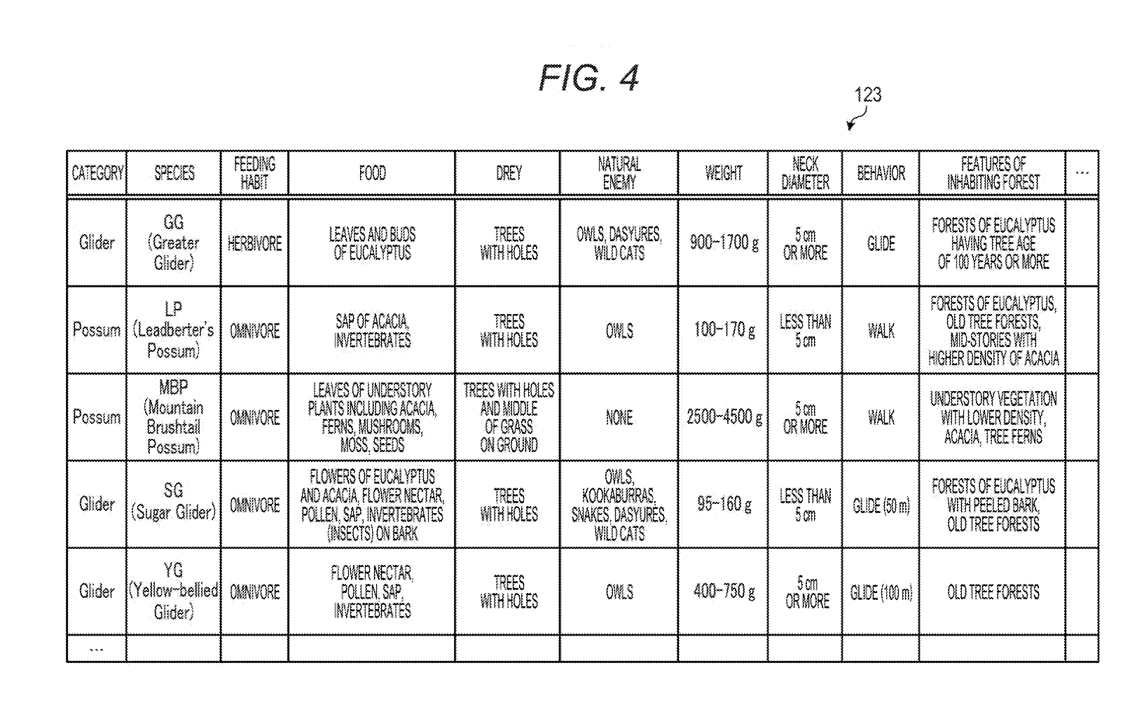

Returning to FIG. 1, the animal feature DB 123 stores features of tree mammals to be determined. FIG. 4 is a diagram illustrating an example of the animal feature DB. As illustrated in FIG. 4, the animal feature DB 123 stores “category”, “species”, “feeding habit”, “food”, “drey”, “natural enemy”, “weight”, “neck diameter”, “behavior”, and “features of inhabiting forest” in association with each other. The information stored in the animal feature DB 123 is, for example, data of each animal or the like acquired by the record acquiring part 131 to be described later.

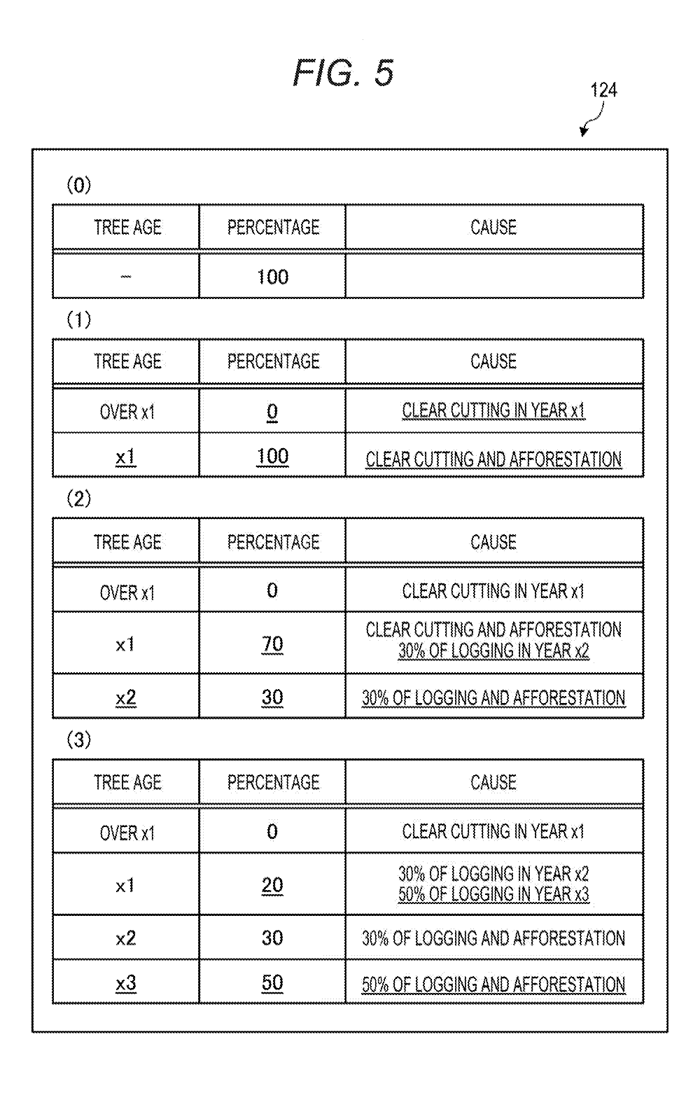

Returning to FIG. 1, the tree age DB 124 stores the percentage of trees by tree age. FIG. 5 is a diagram illustrating an example of the tree age DB according to the first embodiment. As illustrated in FIG. 5, the tree age DB 124 stores “tree age”, “percentage”, and “cause” in association with each other. As in the recording DB 122, for example, the tree age DB 124 has one table for each targeted region ID. The information stored in the tree age DB 124 is, for example, information specified by a tree age specifying part 132 to be described later with reference to the recording DB 122 illustrated in FIG. 3.

In FIG. 5, a table indicated in (0) represents the state before the information is updated by the tree age specifying part 132. A table indicated in (1) of FIG. 5 indicates data updated by the tree age specifying part 132 with reference to a record with years “x1” in the recording DB 122 illustrated in FIG. 3. As illustrated in (1) of FIG. 5, the tree age DB 124 stores that the percentage of trees whose tree age is greater than “x1” has become “0%”, and the percentage of trees whose tree age is “x1” has become “100%”, on the basis of the record that clear cutting and afforestation were performed “x1” years ago.

Similarly, as illustrated in (2) of FIG. 5, the tree age DB 124 stores that the percentage of trees whose tree age is “x1” has become “70%”, and the percentage of trees whose tree age is “x2” has become “30%”, on the basis of the record that 30% of logging and afforestation were performed “x2” years ago. Furthermore, as illustrated in (3) of FIG. 5, the tree age DB 124 stores that the percentage of trees whose tree age is “x1” has become “20%”, and the percentage of trees whose tree age is “x3” has become “50%”, on the basis of the record that 50% of logging and afforestation were performed “x3” years ago. In the present embodiment, it is assumed that logging is performed with priority on trees with higher tree age.

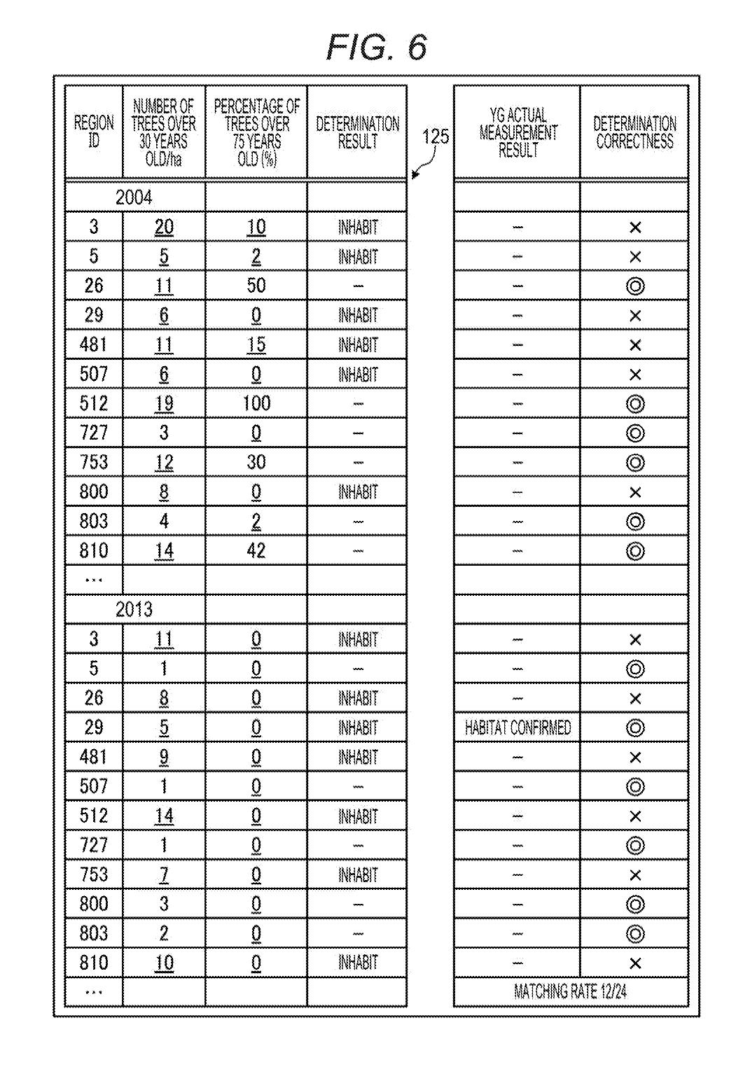

Next, the determination result DB 125 stores the habitat determination result of the target animal in each region. FIG. 6 is a diagram illustrating an example of the determination result DB according to the first embodiment. As illustrated in FIG. 6, the determination result DB 125 stores “region ID”, “number of trees over 30 years old”, “percentage of trees over 75 years old”, and “determination result” in association with each other. For example, the determination result DB 125 has one table for each target animal. The information stored in the determination result DB 125 is input, for example, by the tree age specifying part 132 and a determining part 133 to be described later. The number of trees over 30 years old is an example of the first indicator and the percentage of trees over 75 years old is an example of a second indicator.

In FIG. 6, “determination result” stores the result of determination as to whether or not the target animal inhabits, on the basis of “number of trees over 30 years old” and “percentage of trees over 75 years old” in a region with a targeted region ID. “Number of trees over 30 years old” indicates the number of trees (density) per predetermined area (for example, one hectare) with respect to a tree having a tree hole that can be used as a nest hole by a tree mammal. “Percentage of trees over 75 years old” indicates the percentage of trees having larger holes that can be used as nest holes by larger types of tree mammals. The determination result DB 125 illustrated in FIG. 6 stores the result of a determination process performed for the target animal “YG” for the target regions “12 regions with region IDs “3” to “810”” and the target years “2004 and 2013”.

In FIG. 6, “determination result” indicates the result of determination made by the determining part 133 to be described later, on the basis of “number of trees over 30 years old” and “percentage of trees over 75 years old” as to whether or not the target animal inhabits. FIG. 6 illustrates an example of storing the result of determination that the target animal “YG” inhabits, which has been made by the determining part 133 to be described later when “number of trees over 30 years old” is equal to or greater than five per hectare and “percentage of trees over 75 years old” is less than “30%” in the target region.

In FIG. 6, “YG actual measurement result” indicating whether or not habitat of the target animal “YG” was confirmed as a result of actual measurement in each target region, and “determination correctness” indicating whether or not the determination result and the actual measurement result match are indicated for convenience. As illustrated in FIG. 6, in the present embodiment, the matching rate between the determination result and the actual measurement result is given as 50% (12/24).

Returning to FIG. 1, the control part 130 is a processing unit that supervises the overall process of the habitat determination apparatus 100 and is, for example, a processor or the like. This control part 130 has the record acquiring part 131, the tree age specifying part 132, and the determining part 133. The record acquiring part 131, the tree age specifying part 132, and the determining part 133 are an example of electronic circuits included in the processor and an example of processes executed by the processor.

The record acquiring part 131 acquires information regarding forest logging and the like. For example, the record acquiring part 131 accesses an external database (not illustrated) or the like by way of the communication part 111 and acquires information such as a vegetation map released by the environmental authorities to store the acquired information in the vegetation DB 121. The record acquiring part 131 also accesses an external database (not illustrated) or the like and acquires information such as logging records released by the environmental authorities to store the acquired information in the recording DB 122. Furthermore, the record acquiring part 131 accesses an external database (not illustrated) or the like and acquires information on data of each animal to be determined, to store the acquired information in the animal feature DB 123.

The tree age specifying part 132 specifies the tree age in the target region. For example, the tree age specifying part 132 updates the tree age DB 124 with reference to the causes and percentages of the years stored in the recording DB 122.

The determining part 133 determines whether or not the target animal inhabits the target region. For example, when accepting designation of the target animal, the target region, and the target years from a user (not illustrated) through the communication part 111 or the display operation part 112, the determining part 133 acquires the characteristic of the targeted animal from the animal feature DB 123. In addition, the determining part 133 refers to the tree age DB 124 to specify the number of trees to be targeted and the percentage of trees to be targeted. The determining part 133 then determines whether or not the target animal inhabits the target region or which animal inhabits the target region, on the basis of the specified number of trees and percentage of trees.

For example, the determining part 133 accepts designation of the target animal “YG”, the target regions “12 regions with region IDs “3” to “810””, and the target years “2004 and 2013” from a user (not illustrated). In this case, the determining part 133 first refers to the vegetation DB 121 illustrated in FIG. 2 and determines whether or not the classification of the tree planted in the target region coincides with one of “MA”, “AA” and “SG”. The determining part 133 determines that none of the tree mammals inhabits a region determined that the classification of the tree does not coincide with any of “MA”, “AA”, and “SG”. For example, the determining part 133 determines that none of the tree mammals inhabits the regions with the region IDs “727” and “803” illustrated in FIG. 2.

Next, the determining part 133 refers to the animal feature DB 123 to specify the neck diameter of the target animal as a characteristic of the target animal. In the present embodiment, it is specified that the neck diameter of the target animal “YG” is “5 cm or more”.

Next, the determining part 133 refers to the vegetation DB 121 and the tree age DB 124 for each region to calculate the number of trees having tree age of 30 years or more by multiplying the number of trees in each region by the percentage of trees having tree age of 30 years or more.

For example, it is assumed that, in the tree age DB 124, “x1” represents “year 1950”, “x2” represents “year 1980”, and “x3” represents “year 2010” in the region with the region ID “800”. In this case, at the time point of “2004”, the tree age of a tree with the tree age “x1” is given as “54 years” and the tree age of a tree with the tree age “x2” is given as “24 years”. Since the logging in “x3” has not yet been performed at the time point of “2004”, the percentage of trees with the tree age “x1” is given as “70%” and the percentage of trees with the tree age “x2” is given as “30%”.

At the time pint of “2013”, the tree age of a tree with the tree age “x1” is given as “63 years”, the tree age of a tree with the tree age “x2” is given as “13 years”, and the tree age of a tree with the tree age “x3” is given as “3 years”. Additionally, at the time point of “2013”, the percentage of trees with the tree age “x1” is given as “20%”, the percentage of trees with the tree age “x2” is given as “30%”, and the percentage of trees with the tree age “x3” is given as “50%”.

As described above, the percentage of trees having tree age of 30 years or more in the region with the region ID “800” is “70%” at the time point of “2004” and “20%” at the time point of “2013”. Since the number of trees per hectare of the region ID “800” stored in the vegetation DB 121 is “11”, the number of trees having tree age of 30 years or more at the time point of “2004” is given as “11 trees×70%=7.7 trees”. In addition, the number of trees having tree age of 30 years or more at the time point of “2013” is given as “11 trees×20%=2.2 trees”. In the present embodiment, since it is assumed that decimal places are rounded up, the determining part 133 records “8” in the determination result DB 125 as the number of trees having tree age of 30 years or more at the time point of “2004”. Similarly, the determining part 133 records “3” in the determination result DB 125 as the number of trees having tree age of 30 years or more at the time point of “2013”.

In addition, the percentage of trees having tree age of 75 years or more in the region with the region ID “800” is “0%” at both time points of “2004” and “2013”. Therefore, the determining part 133 records “0%” in the determination result DB 125 as the percentage of trees having tree age of 75 years or more at each time point of “2004” and “2013”.

Then, the determining part 133 determines whether or not the target animal inhabits the target region, on the basis of the specified number of trees having tree age of 30 years or more and percentage of trees having tree age of 75 years or more per hectare. In the present embodiment, when the number of trees having tree age of 30 years or more is equal to or greater than “five” trees/ha and the percentage of trees having tree age of 75 years or more is less than “30%”, it is determined that the target animal YG inhabits. The reason why it is limited to a case where the percentage of trees having tree age of 75 years or more is less than “30%” is that there is a high possibility that trees having tree age of 75 years or more have larger tree holes and the YG, which is a smaller type of tree mammal, no longer can inhabit.

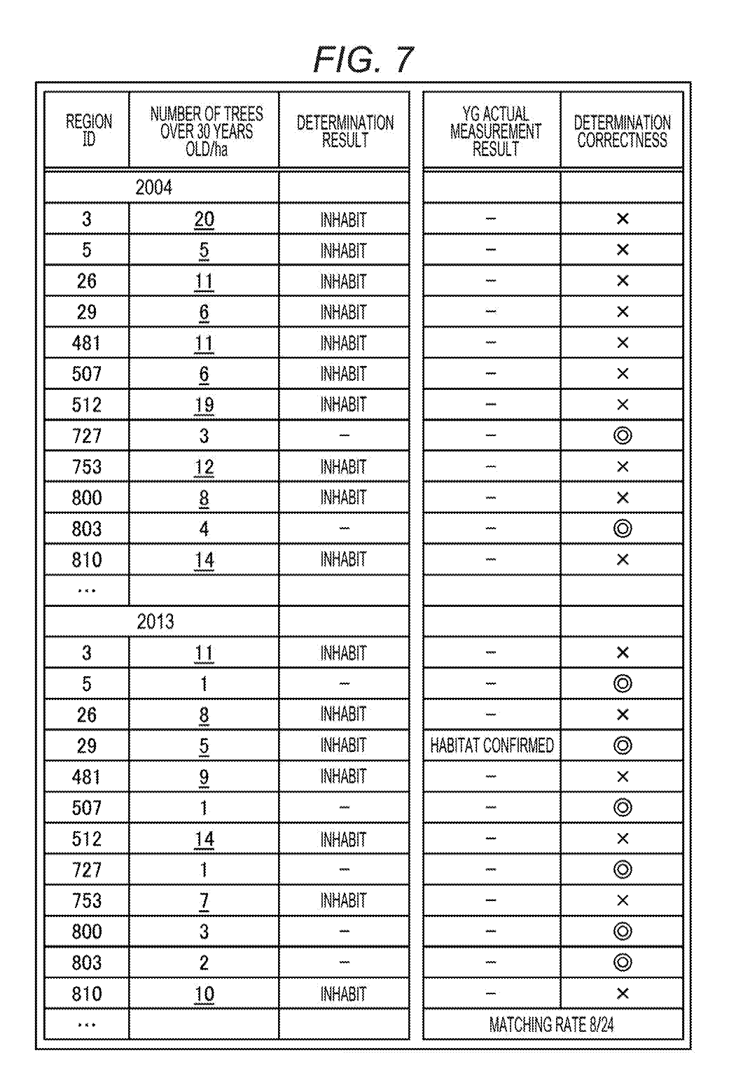

As a result of performing the above process, the determining part 133 stores the determination result, for example, as illustrated in FIG. 6. The comparison between the determination result in the present embodiment and the determination result by a technique according to related art will be described with reference to FIGS. 6 and 7. FIG. 7 is a diagram illustrating an example of the determination technique according to related art. FIG. 7 depicts an example of a technique of determining whether or not the target animal “YG” inhabits the target region, on the basis of the density of trees on which tree holes have been produced, without paying attention to the size of the tree hole according to the tree age.

For example, in the target year “2004”, the region with the region ID “26” was erroneously determined as “inhabit” in the related art illustrated in FIG. 7; in the present embodiment, however, this case is determined to be “not inhabit” because the percentage of trees having tree age of 75 years or more is equal to or greater than “30%”. Also in other target regions, there are cases where determination in the present embodiment for a region erroneously determined in the related art matches with the actual measurement result; consequently, the matching rate in the present embodiment rises from 33.3% (8/24) to 52%. Thus, in the present embodiment, the accuracy of determining whether or not the targeted animal inhabits is reliably improved.

[Flow of Process]

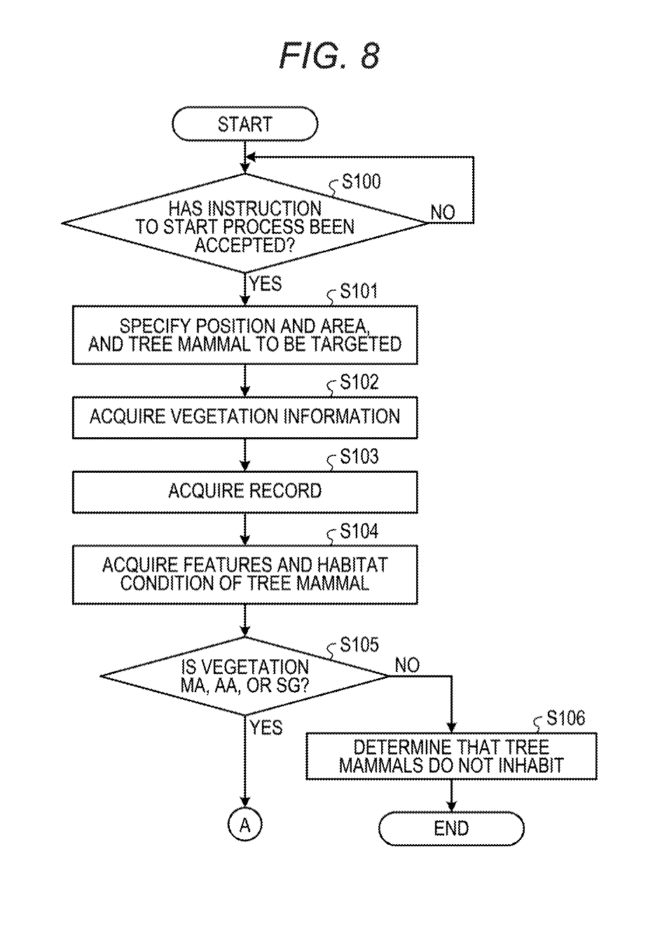

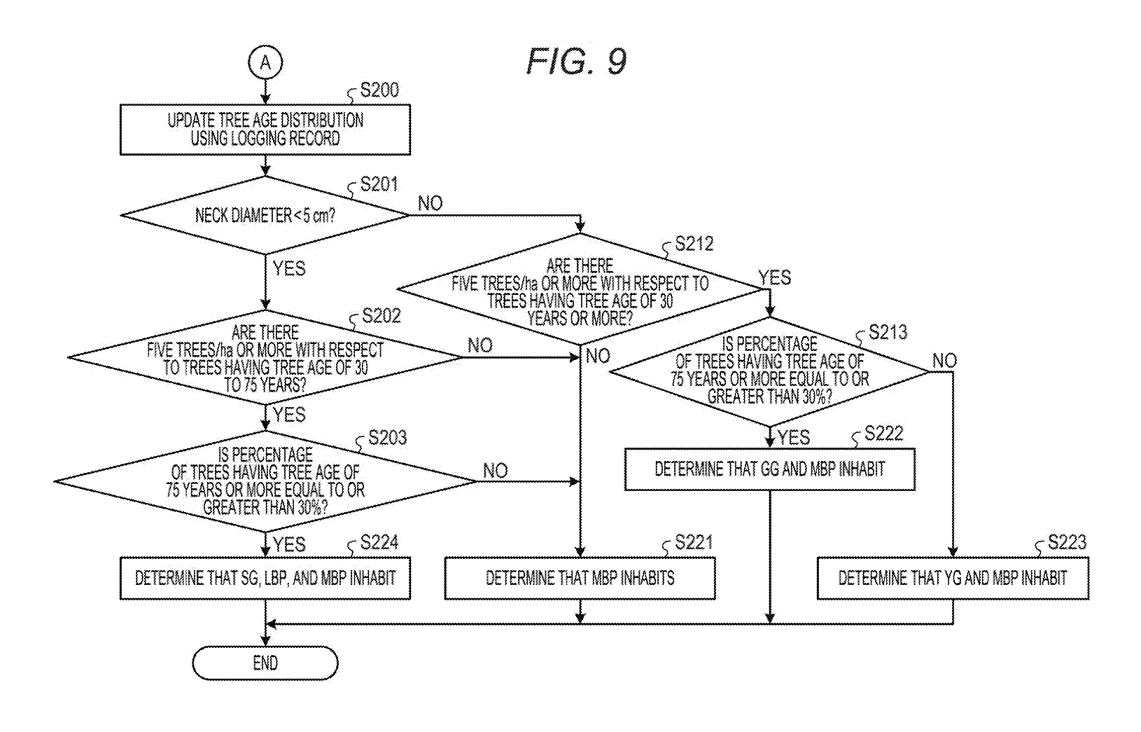

Next, the flow of a process in the present embodiment will be described with reference to FIGS. 8 and 9. The habitat determination apparatus 100 in the present embodiment executes a pre-determination process illustrated in FIG. 8 and a habitat determination process illustrated in FIG. 9.[0042] FIG. 8 is a flowchart illustrating an example of the pre-determination process. As illustrated in FIG. 8, the record acquiring part 131 of the habitat determination apparatus 100 waits until accepting an instruction to start the process from, for example, a terminal of a user (not illustrated) (S100: No). Upon accepting an instruction to start the process (S100: Yes), the record acquiring part 131 specifies the position and area, and the tree mammal to be targeted (S101). For example, the record acquiring part 131 refers to the vegetation DB 121 and the animal feature DB 123 to specify the target animal “YG” and the 12 regions with the region IDs “3” to “810”.

Next, the record acquiring part 131 acquires vegetation information including the number of trees and the tree classification stored in the vegetation DB 121 (S102). The record acquiring part 131 refers to the recording DB 122 to acquire a record such as a logging record (S103). Then, the record acquiring part 131 refers to the animal feature DB 123 to acquire the features and the habitat condition of the tree mammal (S104). For example, it is specified that the target animal “YG” has a neck diameter of “5 cm or more” and inhabits “trees with holes” in an “old tree forests”.

The record acquiring part 131 determines whether or not vegetation in the target region is any of “MA”, “AA”, and “SG” (S105). When determining that the vegetation is other than “MA”, “AA” and “SG” (S105: No), the record acquiring part 131 determines that tree mammals do not inhabit the target region, stores the fact that the target animal does not inhabit in the determination result DB 125 (S106), and terminates the process.

When determining that the vegetation is one of “MA”, “AA” or “SG” (S105: Yes), the record acquiring part 131 proceeds to the habitat determination process illustrated in FIG. 9 through the terminal A. FIG. 9 is a flowchart illustrating an example of the habitat determination process according to the first embodiment. As illustrated in FIG. 9, the determining part 133 first refers to the recording DB 122 to update a tree age distribution recorded in the tree age DB 124 using the logging record (S200).

Next, the determining part 133 refers to the animal feature DB 123 to determine whether or not the neck diameter of the target animal is “less than 5 cm” (S201). When determining that the neck diameter of the target animal is “less than 5 cm” (S201: Yes), the determining part 133 proceeds to S202. When determining that the neck diameter of the target animal is “5 cm or more” (S201: No), the determining part 133 proceeds to S212. For example, when the target animal is “YG”, the determining part 133 proceeds to S212 because the neck diameter thereof is 5 cm or more.

After proceeding to S212, the determining part 133 determines whether or not there are five trees or more per hectare with respect to trees having tree age of 30 years or more in the target region (S212). When determining that there are less than five trees per hectare with respect to trees having tree age of 30 years or more (S212: No), the determining part 133 determines that a mountain brushtail possum (MBP) inhabits the target region (S221). Then, the determining part 133 stores the determination result in the determination result DB 125 and terminates the process. For example, when the target animal is “YG”, the determining part 133 stores the fact that “YG” does “not inhabit” in the determination result DB 125.

On the other hand, when determining that there are five trees or more per hectare with respect to trees having tree age of 30 years or more (S212: Yes), the determining part 133 determines whether or not the percentage of trees having tree age of 75 years or more in the target region is equal to or greater than 30% (S213). When determining that the percentage of trees having tree age of 75 years or more is equal to or greater than 30% (S213: Yes), the determining part 133 determines that the GG and the MBP inhabit the target region, stores the determination result in the determination result DB 125 (S222), and terminates the process. For example, when the target animal is “YG”, the determining part 133 stores the fact that “YG” does “not inhabit” in the determination result DB 125.

On the other hand, when determining that the percentage of trees having tree age of 75 years or more is less than 30% (S213: No), the determining part 133 determines that the YG and the MBP inhabit the target region, stores the determination result in the determination result DB 125 (S223), and terminates the process. For example, when the target animal is “YG”, the determining part 133 stores the fact that “YG” “inhabits” in the determination result DB 125.

Returning to S202, the determining part 133 determines whether or not there are five trees or more per hectare with respect to trees having tree age of 30 years or more and less than 75 years in the target region (S202). When determining that there are less than five trees per hectare with respect to trees having tree age of 30 years or more and less than 75 years (S202: No), the determining part 133 proceeds to S221.

On the other hand, when determining that there are five trees or more per hectare with respect to trees having tree age of 30 years or more and less than 75 years (S202: Yes), the determining part 133 determines whether or not the percentage of trees having tree age of 75 years or more in the target region is equal to or greater than 30% (S203). When determining that the percentage of trees having tree age of 75 years or more is less than 30% (S203: No), the determining part 133 proceeds to S221.

On the other hand, when determining that the percentage of trees having tree age of 75 years or more is equal to or greater than 30% (S203: Yes), the determining part 133 determines that a sugar glider (SG), a lead beater's possum (LBP), and the MBP inhabit the target region. Then, the determining part 133 stores the determination result in the determination result DB 125 (S224) and terminates the process.

[Effects]

As described above, in the habitat determination method according to the present embodiment, a process of calculating the first indicator, which is the number of trees per predetermined area, is performed with respect to a tree allowing a tree mammal to inhabit, and, according to the first indicator and a characteristic of the tree mammal, a process of determining whether or not the tree mammal is inhabitable is performed. With this procedure, habitat determination is made by reflecting the presence or absence of an environment that can be used as a nest hole by a tree mammal, such that the determination accuracy is improved.

In addition, the habitat determination method in the present embodiment performs a process of calculating the first indicator on the basis of information indicating the number of trees having holes with sizes usable as nest holes by a miniature breed of tree mammal. The habitat determination method in the present embodiment performs a process of calculating the second indicator on the basis of information indicating the percentage of trees having holes with sizes usable as nest holes by a larger tree mammal. Then, the habitat determination method in the present embodiment performs a process of determining whether or not a tree mammal is inhabitable, on the basis of the neck diameter of the tree mammal as a characteristic of the tree mammal, the first indicator, and the second indicator. For example, in the habitat determination method in the present embodiment, since habitat determination is made by reflecting the neck diameter of the tree mammal and the size of the nest hole on a tree, the determination accuracy is improved. Note that the information used by the habitat determination apparatus 100 as a characteristic of a tree mammal is not restricted to this example and other information stored in the animal feature DB 123, such as the weight, the food, or the drey of a tree mammal may be used.

Furthermore, the habitat determination method according to the present embodiment performs a process of calculating the tree age of a tree using any one or a plurality of the vegetation map, the logging record, and a forest fire record of the target region, and a process of calculating the first indicator of a tree whose tree age falls within a predetermined range. For example, since the habitat determination method in the present embodiment performs the process using readily available statistical information, as compared with a method of actually measuring the number of witness counts, masses of feces, bite marks on plants, the number of removed individuals, and the like, the cost for determining whether or not a tree mammal inhabits is reduced.

### Second Embodiment

Even in the case of a tree withered due to a forest fire, there is a case where the tree mammal uses a hole on that tree as a hiding place for a period during which there is no risk of collapse of the tree (for example, approximately five years) after the occurrence of the forest fire. Therefore, the present embodiment will describe a configuration in which a habitat determination apparatus 200 (not illustrated) further acquires a record regarding a forest fire, and determines the habitat of the target animal by further reflecting the tree age and the life or death of the tree due to the forest fire.

[Functional Blocks]

First, the functional configuration of the present embodiment will be described. In the following embodiments, the same reference numerals are given to the same parts as the parts illustrated in the above-described drawings, and duplicate explanations will be omitted. The habitat determination apparatus 200 in the present embodiment is realized by a computer similar to the case of the habitat determination apparatus 100 illustrated in FIG. 1, and has a communication part 111, a display operation part 112, and a storage part 220 and a control part 230 not illustrated in FIG. 1.

The storage part 220 has a vegetation DB 121, a recording DB 222, an animal feature DB 123, a tree age DB 224, and a determination result DB 225. The recording DB 222 in the present embodiment further stores a record regarding the forest fire in addition to the record regarding logging stored in the recording DB 122 in the first embodiment. The information stored in the recording DB 222 is, for example, information acquired by the record acquiring part 231 to be described later, which is disclosed in logging records, forest fire records, and the like released by the environmental authorities.

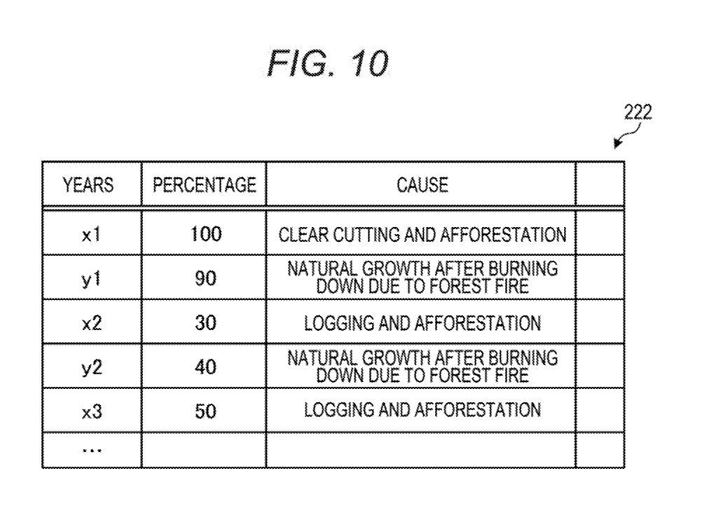

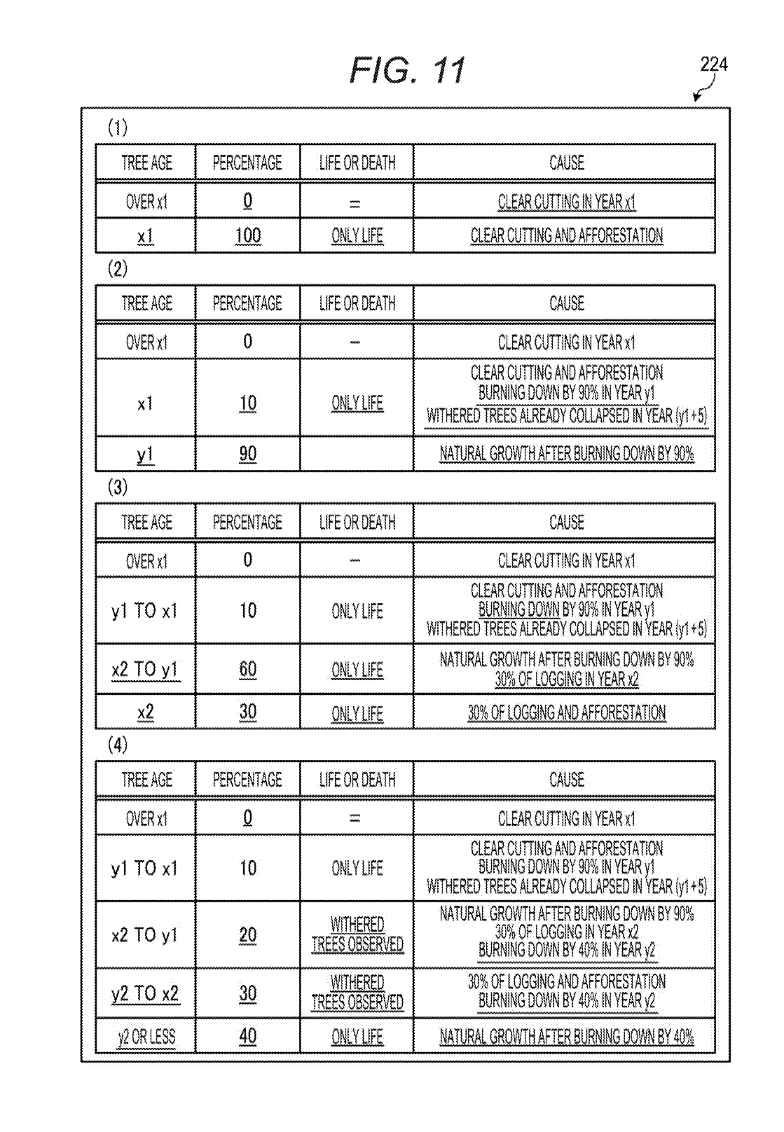

FIG. 10 is a diagram illustrating an example of the recording DB according to the second embodiment. As illustrated in FIG. 10, the recording DB 222 in the present embodiment additionally stores years “y1” and “y2” when a forest fire occurred and the percentage of trees burning down in each year. In the following description, it is assumed that the year “y1” represents “five years or more before” from the target year and a period from the year “y2” to the target year is “less than five years”.

Next, as in the tree age DB 124, the tree age DB 224 stores the percentage of trees by tree age. In the present embodiment, in addition to records regarding logging, the tree age DB 224 further stores the percentage of trees by tree age reflecting records regarding forest fires and the life or death of the trees. FIG. 11 is a diagram illustrating an example of the tree age DB according to the second embodiment. The information stored in the tree age DB 224 is, for example, information specified by a tree age specifying part 232 to be described later with reference to the recording DB 222 illustrated in FIG. 10.

As illustrated in FIG. 11, on the basis of the record regarding a forest fire occurring in the year “y1” recorded in the recording DB 222, the tree age DB 224 stores that “90%” of trees having tree age of “x1” burned down due to the forest fire.

The tree age DB 224 additionally stores whether or not trees withered due to a forest fire remain, depending on the number of years since the forest fire occurred. For example, when a year in which a forest fire occurred is “five years” or more before from the target year, it is supposed that trees withered due to the forest fire have already collapsed and trees that remain are only those that survived without burning down or withering due to the forest fire. On the other hand, when a period from a year in which a forest fire occurred to the target year is less than “five years”, it is supposed that trees that have withered due to the forest fires remain without risk of collapse.

Therefore, as illustrated in FIG. 11, the tree age DB 224 stores that, in the forest fire occurring in the year “y1”, the withered trees do not remain and only inhabited trees last. On the other hand, the tree age DB 224 stores that trees withered due to a forest fire occurring in the year “y2” still remain.

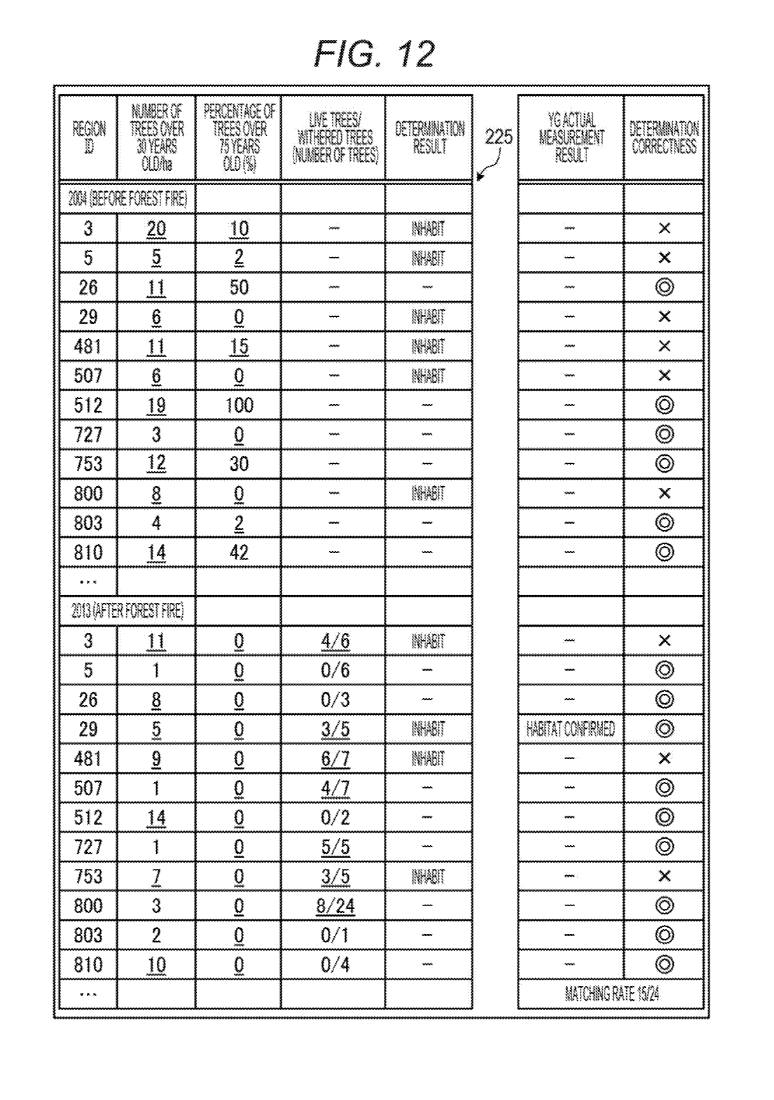

Next, the determination result DB 225 in the present embodiment stores the habitat determination result of the target animal in each region based on the number of trees and the percentage of trees reflecting the tree age and the life or death of the trees due to the forest fire, which are stored in the tree age DB 224. FIG. 12 is a diagram illustrating an example of the determination result DB according to the second embodiment. For example, as in the determination result DB 125, the determination result DB 225 has one table for each target animal. The information stored in the determination result DB 225 is input, for example, by the tree age specifying part 232 and a determining part 233 to be described later.

As illustrated in FIG. 12, the determination result DB 225 further stores “live trees/withered trees” in addition to the information illustrated in the determination result DB 125. In FIG. 12, “live trees/withered trees” indicates the number of living trees and already withered trees among trees having tree age of 20 years or more. “Determination result” in the present embodiment stores the result of determination that the target animal “inhabits”, when the conditions of “number of trees over 30 years old” and “percentage of trees over 75 years old” similar to those of the first embodiment are satisfied and, additionally, there are “three” or more living trees having tree age of 20 years or more. For example, in the target year “2013”, the region ID “26” is determined in the first embodiment that the target animal “YG” “inhabits”; however, in the present embodiment, since living trees having tree age of 20 years or more are less than “three”, it is determined that the target animal “YG” does not inhabit.

Also in FIG. 12, as in FIG. 6, “YG actual measurement result” indicating whether or not habitat of the target animal “YG” was confirmed as a result of actual measurement in each target region, and “determination correctness” indicating whether or not the determination result and the actual measurement result match are indicated for convenience. As illustrated in FIG. 12, in the present embodiment, the matching rate between the determination result and the actual measurement result is given as 62.5% (15/24).

Next, as in the control part 130, the control part 230 is a processing unit that supervises the overall process of the habitat determination apparatus 200 and is, for example, a processor. This control part 230 has a record acquiring part 231, the tree age specifying part 232, and the determining part 233 (not illustrated).

The record acquiring part 231 acquires information regarding forest fires and the like, in addition to the information regarding forest logging. For example, in addition to the process in the record acquiring part 131, the record acquiring part 231 accesses an external database (not illustrated) or the like by way of the communication part 111 and acquires information such as records of forest fires released by the environmental authorities to store the acquired information in the recording DB 222.

The tree age specifying part 232 specifies the tree age in the target region. For example, the tree age specifying part 232 updates the tree age DB 224 with reference to the causes and percentages in the years stored in the recording DB 222.

The determining part 233 determines whether or not the target animal inhabits the target region, further using the percentage of trees by tree age reflecting records of logging, forest fires, and the like stored in the tree age DB 224. The determining part 233 refers to the tree age DB 224 to specify the number of trees and the percentage of trees, and the number of living trees after the forest fire to be targeted. The determining part 233 then determines whether or not the target animal inhabits the target region or which animal inhabits the target region, on the basis of the specified number of trees and percentage of the trees and number of living trees.

For example, with respect to a target region determined that the number of trees having tree age of 30 years or more is equal to or greater than “five” trees/ha and the percentage of trees having tree age of 75 years or more is less than “30%”, the determining part 233 further determines whether or not the number of living trees having tree age of 20 years or more is equal to or greater than “three”. Then, when it is determined that the number of living trees having tree age of 20 years or more is equal to or greater than “three”, the determining part 233 determines that the target animal “inhabits” and stores the determination result in the determination result DB 225.

[Flow of Process]

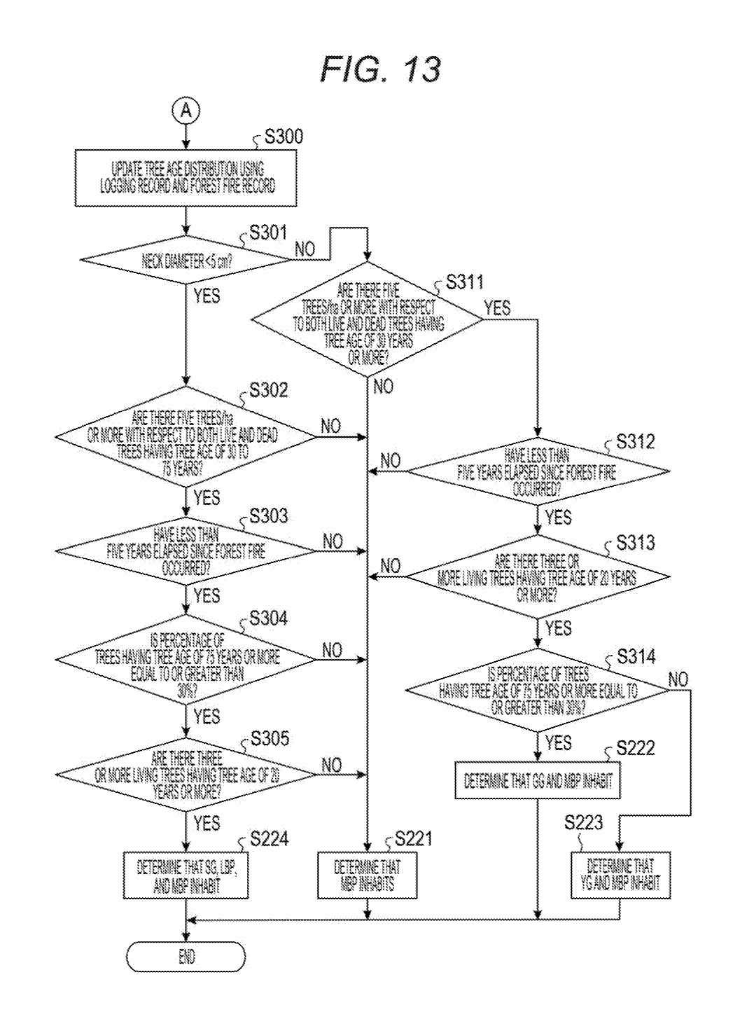

Next, the flow of a habitat determination process in the present embodiment will be described with reference to FIG. 13. The habitat determination process in the present embodiment is executed following the pre-determination process illustrated in FIG. 8.

FIG. 13 is a flowchart illustrating an example of the habitat determination process according to the second embodiment. As illustrated in FIG. 13, the determining part 233 first refers to the recording DB 222 to update a tree age distribution recorded in the tree age DB 224 using the logging record, the forest fire record, and the like (S300).

Next, the determining part 233 refers to the animal feature DB 123 to determine whether or not the neck diameter of the target animal is “less than 5 cm” (S301). When determining that the neck diameter of the target animal is “less than 5 cm” (S301: Yes), the determining part 233 determines whether or not the number of trees having tree age of 30 years or more and less than 75 years in the target region is equal to or greater than five per hectare (S302).

When determining that the sum of living trees and withering trees having tree age of 30 years or more and less than 75 years is less than five per hectare (S302: No), the determining part 233 determines that the MBP inhabits the target region, stores the determination result in the determination result DB 225 (S221), and terminates the process.

On the other hand, when determining that the sum of living trees and withering trees having tree age of 30 years or more and less than 75 years is equal to or greater than five per hectare (S302: Yes), the determining part 233 determines whether or not less than five years have elapsed since the forest fire occurred in the target region (S303). When determining that five years or more have passed since the forest fire occurred (S303: No), the determining part 233 proceeds to S221.

On the other hand, when determining that less than five years have elapsed since the forest fire occurred (S303: Yes), the determining part 233 determines whether or not the percentage of trees having tree age of 75 years or more in the target region is equal to or greater than 30% (S304). When determining that the percentage of trees having tree age of 75 years or more is less than 30% (S304: No), the determining part 233 proceeds to S221.

On the other hand, when determining that the percentage of trees having tree age of 75 years or more is equal to or greater than 30% (S304: Yes), the determining part 233 determines whether or not there are three or more living trees having tree age of 20 years or more in the target region (S305). When determining that there are less than three living trees having tree age of 20 years or more (S305: No), the determining part 233 proceeds to S221.

On the other hand, when determining that there are three or more living trees having tree age of 20 years or more (S305: Yes), the determining part 233 determines that the SG, LBP, and MBP inhabit the target region, stores the determination result in the determination result DB 225 (S224), and terminates the process.

Returning to S301, when determining that the neck diameter of the target animal is “5 cm or more” (S301: No), the determining part 233 determines whether or not the sum of living trees and withering trees having tree age of 30 years or more in the target region is equal to or greater than five per hectare (S311). When determining that the sum of living trees and withering trees having tree age of 30 years or more is less than five per hectare (S311: No), the determining part 233 proceeds to S221.

On the other hand, when determining that the sum of living trees and withering trees having tree age of 30 years or more is equal to or greater than five per hectare (S311: Yes), the determining part 233 determines whether or not less than five years have elapsed since the forest fire occurred in the target region (S312). When determining that five years or more have passed since the forest fire occurred (S312: No), the determining part 233 proceeds to S221.

On the other hand, when determining that less than five years have elapsed since the forest fire occurred (S312: Yes), the determining part 233 determines whether or not there are three or more living trees having tree age of 20 years or more in the target region (S313). When determining that there are less than three living trees having tree age of 20 years or more (S313: No), the determining part 233 proceeds to S221.

On the other hand, when determining that there are three or more living trees having tree age of 20 years or more (S313: Yes), the determining part 233 determines whether or not the percentage of trees having tree age of 75 years or more in the target region is equal to or greater than 30% (S314). When determining that the percentage of trees having tree age of 75 years or more is equal to or greater than 30% (S314: Yes), the determining part 233 determines that the GG and the MBP inhabit the target region (S222). Then, the determining part 233 stores the determination result in the determination result DB 225 and terminates the process.

On the other hand, when determining that the percentage of trees having tree age of 75 years or more is less than 30% (S314: No), the determining part 233 determines that the YG and the MBP inhabit the target region (S223). Then, the determining part 233 stores the determination result in the determination result DB 225 and terminates the process.

[Effects]

As described above, the habitat determination method in the present embodiment performs a process of determining that the tree mammal is not inhabitable when a predetermined number of years have passed since the occurrence of a forest fire specified by the forest fire record. With this procedure, in a case where trees that have withered due to a forest fires exist, habitat determination is made by reflecting whether or not the tree mammal can use the withered tree as a nest hole, such that the determination accuracy is improved.

In the present embodiment, a configuration in which the determining part 233 uses the number of living trees has been described; however, the present invention is not restricted to this embodiment and a configuration to determine whether or not the target animal inhabits, on the basis of the percentage between the living trees and the withered trees may be adopted.

### Third Embodiment

When a tree mammal competes with a different species of tree mammal for food and nest holes, there are cases where a smaller type of tree mammal is not allowed to inhabit a place where a larger type of tree mammal can inhabit. For example, there are cases where the YG, SG, GG, and LBP, which are tree mammals of a type smaller than the MBP, are not allowed to inhabit a place where the MBP, which is a larger type of tree mammal feeding on acacia on understory vegetation, inhabits. Therefore, in the present embodiment, a configuration in which a habitat determination apparatus 300 (not illustrated) determines whether or not the target animal inhabits, on the basis of whether or not a certain amount or more of acacia fed on by the MBP is planted will be described.

[Functional Blocks]

First, the functional configuration of the present embodiment will be described. In the following embodiments, the same reference numerals are given to the same parts as the parts illustrated in the above-described drawings, and duplicate explanations will be omitted. The habitat determination apparatus 300 in the present embodiment is realized by a computer similar to the case of the habitat determination apparatus 100 illustrated in FIG. 1, and has a communication part 111, a display operation part 112, and a storage part 320 and a control part 330 not illustrated in FIG. 1.

The storage part 320 has a vegetation DB 321, a recording DB 222, an animal feature DB 123, a tree age DB 224, and a determination result DB 325. The vegetation DB 321 in the present embodiment further stores “vegetation amount of acacia” in addition to the information stored in the vegetation DB 121 in the first embodiment. As in the vegetation DB 121, the information stored in the vegetation DB 321 is, for example, information acquired by a record acquiring part 331 to be described later, which is disclosed in a vegetation map or the like released by the environmental authorities.

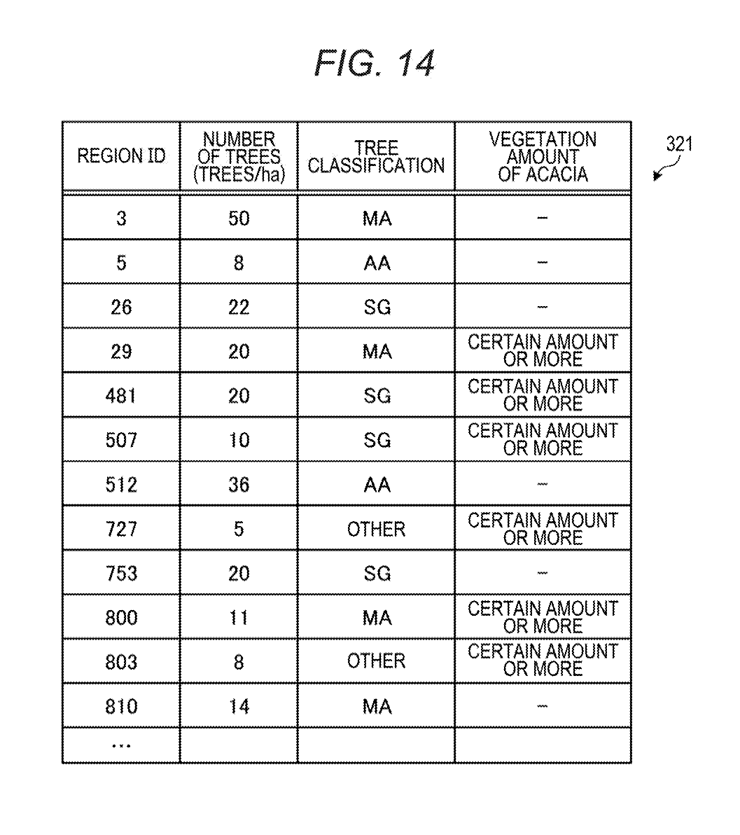

FIG. 14 is a diagram illustrating an example of the vegetation DB according to the third embodiment. As illustrated in FIG. 14, the vegetation DB 321 in the present embodiment further stores whether or not the vegetation amount of acacia is “certain amount or more”. For example, the vegetation DB 321 stores that the vegetation amount of acacia is “certain amount or more” in the region with the region ID “29”, and the vegetation amount of acacia is not “certain amount or more” in the region with the region ID “26”.

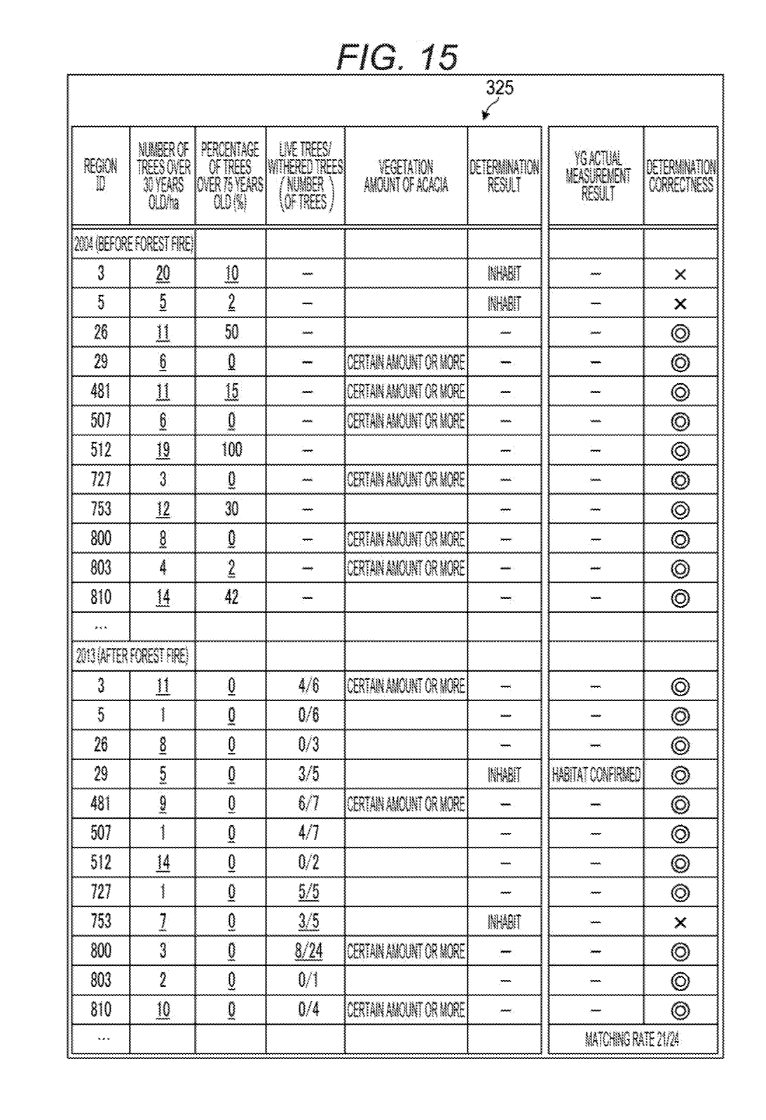

Next, in addition to the information stored in the determination result DB 225, the determination result DB 325 in the present embodiment further stores the vegetation amount of acacia and the habitat determination result of the target animal reflecting the vegetation amount of acacia. FIG. 15 is a diagram illustrating an example of the determination result DB according to the third embodiment. For example, as in the determination result DB 225, the determination result DB 325 has one table for each target animal. The information stored in the determination result DB 325 is input, for example, by the tree age specifying part 232 and a determining part 333 to be described later.

As illustrated in FIG. 15, in addition to the information illustrated in the determination result DB 225, the determination result DB 325 further stores “vegetation amount of acacia” extracted from the vegetation DB 321. “Determination result” in the present embodiment further stores the result of determination that the target animal “inhabits” when conditions similar to those in the second embodiment are satisfied and the vegetation amount of acacia is not “certain amount or more”. For example, in the target year “2013”, the region ID “3” is determined in the second embodiment that the target animal “YG” “inhabits”; however, in the present embodiment, since the vegetation amount of acacia is “certain amount or more”, it is determined that the target animal “YG” does not inhabit.

Also in FIG. 15, as in FIG. 12, “YG actual measurement result” indicating whether or not habitat of the target animal “YG” was confirmed as a result of actual measurement in each target region, and “determination correctness” indicating whether or not the determination result and the actual measurement result match are indicated for convenience. As illustrated in FIG. 15, in the present embodiment, the matching rate between the determination result and the actual measurement result is given as 87.5% (21/24).

Next, as in the control part 130, the control part 330 is a processing unit that supervises the overall process of the habitat determination apparatus 300 and is, for example, a processor. This control part 330 has a record acquiring part 331, the tree age specifying part 232, and the determining part 333 (not illustrated).

In addition to the information acquired by the record acquiring part 231, the record acquiring part 331 further acquires information regarding the vegetation amount of acacia. For example, in addition to the process in the record acquiring part 231, the record acquiring part 331 accesses an external database (not illustrated) or the like by way of the communication part 111 and acquires information such as the vegetation situation of acacia released by the environmental authorities to store the acquired information in the vegetation DB 321.

The determining part 333 determines whether or not the target animal inhabits the target region, further using the vegetation amount of acacia stored in the vegetation DB 321. With reference to the vegetation DB 321, the determining part 333 specifies whether or not the vegetation amount of acacia is the certain amount or more, in addition to the number of trees and the percentage of trees, and the number of living trees after the forest fire to be targeted. The determining part 333 then determines whether or not the target animal inhabits the target region or which animal inhabits the target region, on the basis of the specified number of trees and percentage of trees, number of living trees, and vegetation amount of acacia.

For example, the determining part 333 specifies the vegetation amount of acacia in a region determined that the number of trees having tree age of 30 years or more is equal to or greater than “five” trees/ha, the percentage of trees having tree age of 75 years or more is less than “30%”, and the number of living trees having tree age of 20 years or more is equal to or greater than “three”. Then, when it is determined that the vegetation amount of acacia is “certain amount or more”, the determining part 333 determines that the MPB “inhabits” and stores the determination result in the determination result DB 325. Meanwhile, when it is determined that the vegetation amount of acacia is not “certain amount or more”, the determining part 333 determines that a tree mammal other than the MPB “inhabits” and stores the determination result in the determination result DB 325.

[Flow of Process]

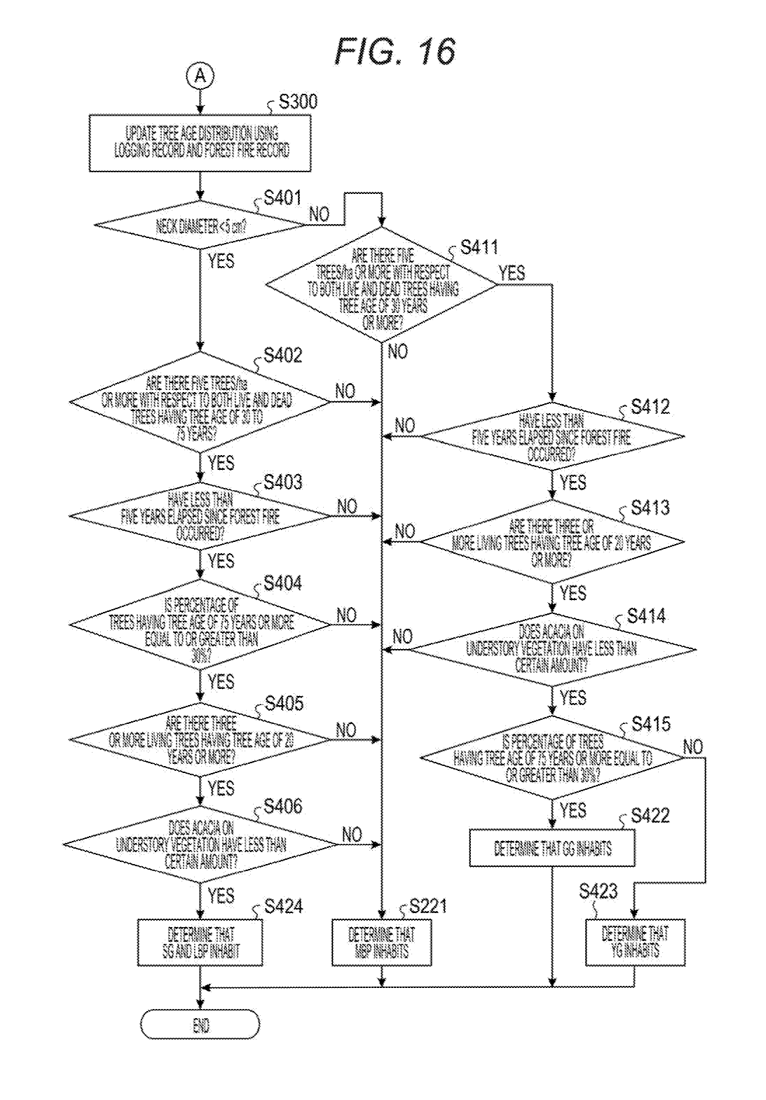

Next, the flow of a habitat determination process in the present embodiment will be described with reference to FIG. 16. The habitat determination process in the present embodiment is also executed following the pre-determination process illustrated in FIG. 8.

FIG. 16 is a flowchart illustrating an example of the habitat determination process according to the third embodiment. As illustrated in FIG. 16, the determining part 333 first refers to the recording DB 222 to update a tree age distribution recorded in the tree age DB 224 using the logging record (S300).

Next, the determining part 333 refers to the animal feature DB 123 to determine whether or not the neck diameter of the target animal is “less than 5 cm” (S401). When determining that the neck diameter of the target animal is “less than 5 cm” (S401: Yes), the determining part 333 determines whether or not the number of trees having tree age of 30 years or more and less than 75 years in the target region is equal to or greater than five per hectare (S402).

When determining that the sum of living trees and withering trees having tree age of 30 years or more and less than 75 years is less than five per hectare (S402: No), the determining part 333 determines that the MBP inhabits the target region, stores the determination result in the determination result DB 325 (S221), and terminates the process.

On the other hand, when determining that the sum of living trees and withering trees having tree age of 30 years or more and less than 75 years is equal to or greater than five per hectare (S402: Yes), the determining part 333 determines whether or not less than five years have elapsed since the forest fire occurred in the target region (S403). When determining that five years or more have passed since the forest fire occurred (S403: No), the determining part 333 proceeds to S221.

On the other hand, when determining that less than five years have elapsed since the forest fire occurred (S403: Yes), the determining part 333 determines whether or not the percentage of trees having tree age of 75 years or more in the target region is equal to or greater than 30% (S404). When determining that the percentage of trees having tree age of 75 years or more is less than 30% (S404: No), the determining part 333 proceeds to S221.

On the other hand, when determining that the percentage of trees having tree age of 75 years or more is equal to or greater than 30% (S404: Yes), the determining part 333 determines whether or not there are three or more living trees having tree age of 20 years or more in the target region (S405). When determining that there are less than three living trees having tree age of 20 years or more (S405: No), the determining part 333 proceeds to S221.

On the other hand, when determining that there are three or more living trees having tree age of 20 years or more (S405: Yes), the determining part 333 determines whether or not acacia on understory vegetation has less than the certain amount (S406). When determining that acacia on understory vegetation has equal to or greater than the certain amount (S406: No), the determining part 333 proceeds to S221.

On the other hand, when determining that acacia on understory vegetation has less than the certain amount (S406: Yes), the determining part 333 determines that the SG and the LBP inhabit the target region, stores the determination result in the determination result DB 325 (S424), and terminates the process.

Returning to S401, when determining that the neck diameter of the target animal is “5 cm or more” (S401: No), the determining part 333 determines whether or not the sum of living trees and withering trees having tree age of 30 years or more in the target region is equal to or greater than five per hectare (S411). When determining that the sum of living trees and withering trees having tree age of 30 years or more is less than five per hectare (S411: No), the determining part 333 proceeds to S221.

On the other hand, when determining that the sum of living trees and withering trees having tree age of 30 years or more is equal to or greater than five per hectare (S411: Yes), the determining part 333 determines whether or not less than five years have elapsed since the forest fire occurred in the target region (S412). When determining that five years or more have passed since the forest fire occurred (S412: No), the determining part 333 proceeds to S221.

On the other hand, when determining that less than five years have elapsed since the forest fire occurred (S412: Yes), the determining part 333 determines whether or not there are three or more living trees having tree age of 20 years or more in the target region (S413). When determining that there are less than three living trees having tree age of 20 years or more (S413: No), the determining part 333 proceeds to S221.

On the other hand, when determining that there are three or more living trees having tree age of 20 years or more (S413: Yes), the determining part 333 determines whether or not acacia on understory vegetation has less than the certain amount (S414). When determining that acacia on understory vegetation has equal to or greater than the certain amount (S414: No), the determining part 333 proceeds to S221.

On the other hand, when determining that acacia on understory vegetation has less than the certain amount (S414: Yes), the determining part 333 determines whether or not the percentage of trees having tree age of 75 years or more in the target region is equal to or greater than 30% (S415). When determining that the percentage of trees having tree age of 75 years or more is equal to or greater than 30% (S415: Yes), the determining part 333 determines that the GG inhabits the target region, stores the determination result in the determination result DB 325 (S422), and terminates the process.

On the other hand, when determining that the percentage of trees having tree age of 75 years or more is less than 30% (415: No), the determining part 333 determines that the YG inhabits the target region, stores the determination result in the determination result DB 325 (S423), and terminates the process.

[Effects]

As described above, in determining whether or not a first animal and a second animal are inhabitable, the habitat determination method according to the present embodiment performs a process of determining that the first animal inhabits and the second animal does not inhabit, when there is a certain amount or more of plants serving as feed of the first animal. With this procedure, habitat determination is made on the basis of the competitive relationship among tree mammals, such that the determination accuracy is improved.

### Fourth Embodiment

Although the embodiments of the present invention have been described so far, the present invention may be carried out in various different forms in addition to the above-described embodiments.

[System]

Among all the processes described in the present embodiments, all or a part of the processes described as being automatically performed can be manually performed. Alternatively, all or a part of the processes described as being performed manually can be automatically performed by a method. Besides, any information indicated in this document or the drawings, including the processing procedures, control procedures, specific names, and various sorts of data and parameters can be arbitrarily modified unless otherwise noted.

The respective constituent elements of each apparatus illustrated in the drawings are functionally conceptual and do not necessarily have to be physically configured as illustrated in the drawings. For example, specific forms of distribution and integration of the respective apparatuses are not restricted to the forms illustrated in the drawings. This means that all or a part of the apparatus can be configured by being functionally or physically distributed and integrated in arbitrary units according to various sorts of loads and usage situations and the like. For example, the recording DB 122 and the animal feature DB 123 may be integrated, while the determining part 133 may be distributed to a processing unit that specifies the percentage of trees by tree age and a processing unit that determines whether or not the target animal inhabits. Furthermore, all or an arbitrary part of respective processing functions performed in each apparatus may be implemented by a central processing unit (CPU) and a program analyzed and executed by the CPU, or may be implemented as hardware by wired logic.

[Habitat Determination Program]

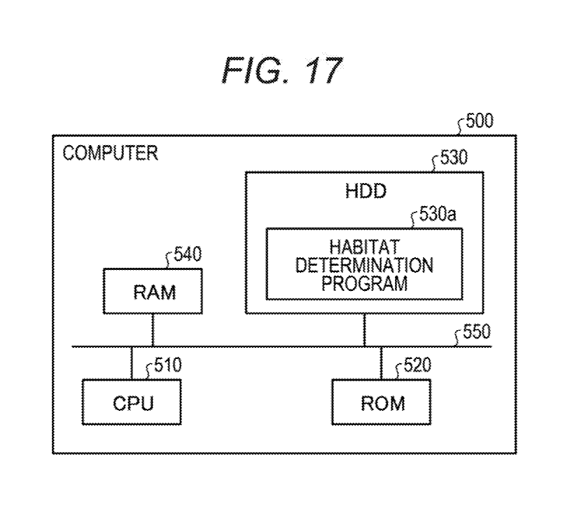

Various sorts of processes of the habitat determination apparatus 100 described in the above embodiment can be implemented by executing a program prepared in advance on a computer system such as a personal computer or a workstation. Therefore, in the following, an example of a computer that executes a habitat determination program having the same functions as the functions of the habitat determination apparatus 100, 200 or 300 described in the above embodiments will be described with reference to FIG. 17. FIG. 17 is a diagram illustrating an example of a computer that executes the habitat determination program.

As illustrated in FIG. 17, a computer 500 has a CPU 510, a ROM 520, a hard disk drive (HDD) 530, and a RAM 540. These respective instruments 510 to 540 are coupled via a bus 550.

The ROM 520 stores a basic program such as an operating system (OS). The HDD 530 stores in advance a habitat determination program 530a that exercises the same functions as the functions of the record acquiring part 131, the tree age specifying part 132, and the determining part 133 indicated in the above embodiment. The HDD 530 also stores various sorts of DBs and attribute value information stored in the storage part 120.

Then, the CPU 510 reads the habitat determination program 530a from the HDD 530 to execute.

Additionally, the CPU 510 reads various sorts of DBs and attribute value information from the HDD 530 to retain in the RAM 540. Furthermore, the CPU 510 executes the habitat determination program 530a using various sorts of DBs and attribute value information retained in the RAM 540. All the data does not need to be regularly retained in the RAM 540. Simply, data used for a process is retained in the RAM 540.

The above-described habitat determination program 530a may not necessarily be stored in the HDD 530 initially.

For example, the program may be stored in a “portable physical medium” such as a flexible disk (FD), a compact disk read only memory (CD-ROM), a digital versatile disc (DVD), a magneto-optical disk, or an integrated circuit (IC) card inserted in the computer 500. Then, the computer 500 reads the program from these media to execute.

Furthermore, the program may be stored in “another computer (or a server)” or the like connected to the computer 500 via a public line, the Internet, a local area network (LAN), a wide area network (WAN), or the like. Then, the computer 500 reads the program from this computer or server to execute.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A habitat determination method comprising:
calculating, by a computer, a first indicator, which is a number of trees per predetermined area with respect to a tree which allows tree mammals to inhabit; and
determining, by the computer, according to the calculated first indicator and a characteristic of each tree mammal, whether or not each tree mammal is inhabitable.

2. The habitat determination method according to claim 1, wherein
the calculating calculates the first indicator on the basis of information indicating the number of trees having holes with sizes usable as nest holes by a miniature tree mammal among the tree mammals, and calculates the second indicator on the basis of information indicating a percentage of trees having holes with sizes usable as nest holes by a tree mammal greater than the miniature tree mammal among the tree mammals; and
the determining determines whether or not each tree mammal is inhabitable, on the basis of a neck diameter of each tree mammal as the characteristic of each tree mammal, the first indicator, and the second indicator.

3. The habitat determination method according to claim 1, wherein the calculating calculates tree age of a tree on the basis of any one or a plurality of a vegetation map, a logging record, and a forest fire record of a target region, and calculates the first indicator of a tree whose tree age falls within a predetermined range.

4. The habitat determination method according to claim 3, wherein the determining determines that each tree mammal is not inhabitable when a predetermined number of years have passed since occurrence of a forest fire which is specified by the forest fire record.

5. The habitat determination method according to claim 1, wherein, in determining whether or not a first animal and a second animal having a competitive relationship are inhabitable, the determining determines that the first animal inhabits and the second animal does not inhabit, when there is a certain amount or more of plants serving as feed of the first animal.

6. An information processing apparatus comprising:
a memory; and
a processor coupled to the memory and configured to:
calculate a first indicator, which is a number of trees per predetermined area with respect to a tree which allows tree mammals to inhabit; and
determine, according to the calculated first indicator and a characteristic of each tree mammal, whether or not each tree mammal is inhabitable.

7. The information processing apparatus according to claim 6, wherein
the processor is configured to:
calculate the first indicator on the basis of information indicating the number of trees having holes with sizes usable as nest holes by a miniature tree mammal among the tree mammals;
calculate the second indicator on the basis of information indicating a percentage of trees having holes with sizes usable as nest holes by a tree mammal greater than the miniature tree mammal among the tree mammals; and
determine whether or not each tree mammal is inhabitable, on the basis of a neck diameter of each tree mammal as the characteristic of each tree mammal, the first indicator, and the second indicator.

8. The information processing apparatus according to claim 6, wherein the processor calculates tree age of a tree on the basis of any one or a plurality of a vegetation map, a logging record, and a forest fire record of a target region, and calculates the first indicator of a tree whose tree age falls within a predetermined range.

9. The information processing apparatus according to claim 8, wherein the processor determines that each tree mammal is not inhabitable when a predetermined number of years have passed since occurrence of a forest fire which is specified by the forest fire record.

10. The information processing apparatus according to claim 6, wherein, in determining whether or not a first animal and a second animal having a competitive relationship are inhabitable, the processor determines that the first animal inhabits and the second animal does not inhabit, when there is a certain amount or more of plants serving as feed of the first animal.

11. A non-transitory computer-readable recording medium having stored therein a habitat determination program for causing a computer to execute a process comprising:
calculating a first indicator, which is a number of trees per predetermined area with respect to a tree which allows tree mammals to inhabit; and
determining, according to the calculated first indicator and a characteristic of each tree mammal, whether or not each tree mammal is inhabitable.

12. The non-transitory computer-readable recording medium according to claim 11, wherein
the calculating calculates the first indicator on the basis of information indicating the number of trees having holes with sizes usable as nest holes by a miniature tree mammal among the tree mammals, and calculates the second indicator on the basis of information indicating a percentage of trees having holes with sizes usable as nest holes by a tree mammal greater than the miniature tree mammal among the tree mammals; and
the determining determines whether or not each tree mammal is inhabitable, on the basis of a neck diameter of each tree mammal as the characteristic of each tree mammal, the first indicator, and the second indicator.

13. The non-transitory computer-readable recording medium according to claim 11, wherein the calculating calculates tree age of a tree on the basis of any one or a plurality of a vegetation map, a logging record, and a forest fire record of a target region, and calculates the first indicator of a tree whose tree age falls within a predetermined range.

14. The non-transitory computer-readable recording medium according to claim 13, wherein the determining determines that each tree mammal is not inhabitable when a predetermined number of years have passed since occurrence of a forest fire which is specified by the forest fire record.

15. The non-transitory computer-readable recording medium according to claim 11, wherein, in determining whether or not a first animal and a second animal having a competitive relationship are inhabitable, the determining determines that the first animal inhabits and the second animal does not inhabit, when there is a certain amount or more of plants serving as feed of the first animal.
